(19) United States
(12) Patent Application Publication
(10) Pub. No.: US 2023/0082016 A1
(43) Pub. Date: Mar. 16, 2023

(54) MASK FOR NON-CONTACT RESPIRATORY MONITORING

(71) Applicant: Covidien LP, Mansfield, MA (US)

(72) Inventors: Dean MONTGOMERY, Edinburgh (GB); Paul S. ADDISON, Edinburgh (GB); Dominique JACQUEL, Edinburgh (GB)

(21) Appl. No.: 17/814,798

(22) Filed: Jul. 25, 2022

Related U.S. Application Data
(60) Provisional application No. 63/244,331, filed on Sep. 15, 2021.

(51) Int. Cl. A61B 5/08; A61B 5/00

(52) U.S. Cl. CPC A61B 5/0816; A61B 5/0803; A61B 5/0077; A61B 5/0806

(57) ABSTRACT

Methods and systems for non-contact monitoring of a patient to determine a respiratory parameter such as respiration rate. The systems and methods receive a depth signal from the patient to determine patient movement indicative of respiration. The methods include analyzing multiple regions in a region of interest (ROI) to determine whether or not respiration is occurring in the analyzed region, and preparing a mask with the regions determined to have respiration. The mask is used to determine the respiratory parameter of the patient in the masked ROI.

### CROSS-REFERENCE TO RELATED APPLICATIONS

The present application claims benefit of priority to U.S. Provisional Patent Application No. 63/244,331, entitled “MASK FOR NON-CONTACT RESPIRATORY MONITORING” and filed on Sep. 15, 2021, which is specifically incorporated by reference herein for all that it discloses or teaches.

### BACKGROUND

Many conventional medical monitors require attachment of a sensor to a patient in order to detect physiologic signals from the patient and transmit detected signals through a cable to the monitor. These monitors process the received signals and determine vital signs such as the patient's pulse rate, respiration rate, and arterial oxygen saturation. For example, a pulse oximeter is a finger sensor that may include two light emitters and a photodetector. The sensor emits light into the patient's finger and transmits the detected light signal to a monitor. The monitor includes a processor that processes the signal, determines vital signs (e.g., pulse rate, respiration rate, arterial oxygen saturation), and displays the vital signs on a display.

Other monitoring systems include other types of monitors and sensors, such as electroencephalogram (EEG) sensors, blood pressure cuffs, temperature probes, air flow measurement devices (e.g., spirometer), and others. Some wireless, wearable sensors have been developed, such as wireless EEG patches and wireless pulse oximetry sensors.

Video-based monitoring is a new field of patient monitoring that uses a remote video camera to detect physical attributes of the patient, such as respiratory parameters including respiration rate, tidal volume, minute volume, effort to breathe, activity, etc. This type of monitoring may also be called “non-contact” monitoring in reference to the remote video sensor, which does not contact the patient.

### SUMMARY

The present disclosure is directed to methods for non-contact monitoring of a patient to determine respiratory parameters such as respiration rate, tidal volume, minute volume, oxygen saturation, and other parameters such as motion and activity. The methods determine regions in the region of interest (ROI) in the field of view of the monitoring system that have active respiration and from those regions develop a mask that is applied to the ROI to focus the respiratory monitoring and inhibit collection of data noise.

One particular embodiment described herein is a method for determining a respiratory parameter of a patient, by determining depth data between a non-contact patient monitoring system and the patient, over time, in a region of interest (ROI) on the patient, the ROI having multiple regions and each region having at least one depth data. Each of the multiple regions in the ROI are analyzed to create a background image overlay from the depth data, and each of the multiple regions in the ROI are analyzed to determine whether respiration is occurring in the analyzed region, and dependent on respiration not occurring in an analyzed region, not including that analyzed region in a mask calculation, and dependent on respiration occurring in an analyzed region, including that analyzed region in the mask calculation. The method also includes preparing a mask comprising a plurality of regions from the mask calculation and extracting the depth data corresponding to respiration, using the depth data corresponding to respiration to create a visual respiration overlay, and combining the visual respiration overlay with the background image overlay to produce a respiration image.

Another particular embodiment described herein is a method of determining a respiration region of interest (ROI) of a patient, by obtaining depth data from a non-contact patient monitoring system for a patient in a field of view (FOV), analyzing the depth data from multiple regions in the FOV to create a background image from the depth data, and analyzing the depth data from the multiple regions the FOV to determine whether respiration is occurring, and dependent on respiration not occurring in an analyzed region, not including that analyzed region in a mask calculation, and dependent on respiration occurring in an analyzed region, including that analyzed region in the mask calculation. The method further includes preparing a mask from the mask calculation and extracting the depth data corresponding to respiration, using the depth data corresponding to respiration to create a visual respiration overlay, and combining the visual respiration overlay with the background image to produce a respiration image.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Other embodiments are also described and recited herein.

### BRIEF DESCRIPTION OF THE DRAWING

FIG. 1 is a schematic diagram of an example non-contact patient monitoring system according to various embodiments described herein.

FIG. 2A and FIG. 2B are schematic diagrams showing two embodiments using the example non-contact patient monitoring system of FIG. 1.

FIG. 3 is a block diagram of a computing device, a server, and an image capture device according to various embodiments described herein.

FIGS. 4A, 4B, 4C, 4D and 4E are a sequence of images of a patient being monitored by a non-contact patient monitoring system.

FIGS. 5A, 5B, 5C and 5D are images of a patient being monitored showing an example progression of visualization with a mask applied according to this disclosure.

FIG. 6 is a stepwise method of an example method for using a mask for a non-contact patient monitoring system according to various embodiments described herein.

FIG. 7 is another stepwise method of an example method for using a mask for a non-contact patient monitoring system according to various embodiments described herein.

FIG. 8 is an image of a patient being monitored showing a mask applied to the monitored region, as well as a graph of the monitored respiratory parameter.

### DETAILED DESCRIPTION

As described above, the present disclosure is directed to medical monitoring, and in particular, non-contact, video-based monitoring of respiratory parameters, including respiration rate, tidal volume, minute volume, oxygen saturation, and other parameters such as motion or activity. Systems and methods are described for receiving a video signal view of a patient, identifying a physiologically relevant area within the video image (such as a patient's forehead or chest), extracting a distance or depth signal from the relevant area, filtering those signals to determine a mask focused on respiration, and applying the mask to the depth signal data to filter out unwanted signals such as noise.

The signals are detected by a camera or camera system that views but does not contact the patient. With appropriate selection, filtering, and manipulation of the data from the signals detected by the camera, the physiologic contribution by the detected depth signal can be isolated and measured. This approach has the potential to improve signal accuracy, along with many other potential advantages discussed below.

Non-contact or remote monitoring, such as video-based monitoring, can deliver significant benefits over contact monitoring. Some video-based monitoring can reduce cost and waste by reducing use of disposable contact sensors, replacing them with reusable camera systems. Video monitoring may also reduce the spread of infection, by reducing physical contact between caregivers and patients. Video cameras can improve patient mobility and comfort, by freeing patients from wired tethers or bulky wearable sensors. In some cases, these systems can also save time for caregivers, who no longer need to reposition, clean, inspect, or replace contact sensors.

One challenge with video monitoring is motion or movement of the patient. The problem can be illustrated with the example of conventional, contact, pulse oximetry, which utilizes a sensor having two light emitters and a photodetector. The sensor is placed in contact with the patient, such as by clipping or adhering the sensor around a finger, toe, or ear of the patient. The sensor's emitters emit light of two particular wavelengths into the patient's tissue, and the photodetector detects the light after it is reflected or transmitted through the tissue. The detected light signal, called a photoplethysmogram (PPG), modulates with the patient's heartbeat, as each arterial pulse passes through the monitored tissue and affects the amount of light absorbed or scattered. Movement of the patient can interfere with this contact-based oximetry, introducing noise into the PPG signal due to compression of the monitored tissue, disrupted coupling of the sensor to the finger, pooling or movement of blood, exposure to ambient light, and other factors. Modern pulse oximeters use filtering algorithms to remove noise introduced by motion and to continue to monitor the pulsatile arterial signal.

However, movement in non-contact pulse oximetry creates different complications, due to the extent of movement possible between the patient and the camera. Because the camera is remote from the patient, the patient may move toward or away from the camera, creating a moving frame of reference, or may rotate with respect to the camera, effectively morphing the region that is being monitored. Thus, the monitored tissue can change morphology within the image frame over time. This freedom of motion of the monitored tissue with respect to the detector introduces new types of motion noise into the video-based signals.

The present disclosure describes methods for addressing this motion noise and other data or signal noise.

The present disclosure describes methods for non-contact monitoring of a patient to determine respiratory parameters such as respiration rate, tidal volume, minute volume, oxygen saturation, and other parameters such as motion and activity. The systems and methods receive a video signal from the patient and from that extract a distance or depth signal from the relevant area to calculate the parameter(s) from the depth signal.

The depth sensing feature provides a measurement of the distance or depth between the detection system and the patient. One or two video cameras may be used to determine the depth, and change in depth, from the system to the patient. When two cameras, set at a fixed distance apart, are used, they offer stereo vision due to the slightly different perspectives of the scene from which distance information is extracted. When distinct features are present in the scene, the stereo image algorithm can find the locations of the same features in the two image streams. However, if an object is featureless (e.g., a smooth surface with a monochromatic color), then the depth camera system has difficulty resolving the perspective differences. By including an image projector to project features (e.g., in the form of dots, pixels, etc.) onto the scene, this projected feature can be monitored over time to produce an estimate of changing distance or depth.

In the following description, reference is made to the accompanying drawing that forms a part hereof and in which is shown by way of illustration at least one specific embodiment. The following description provides additional specific embodiments. It is to be understood that other embodiments are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

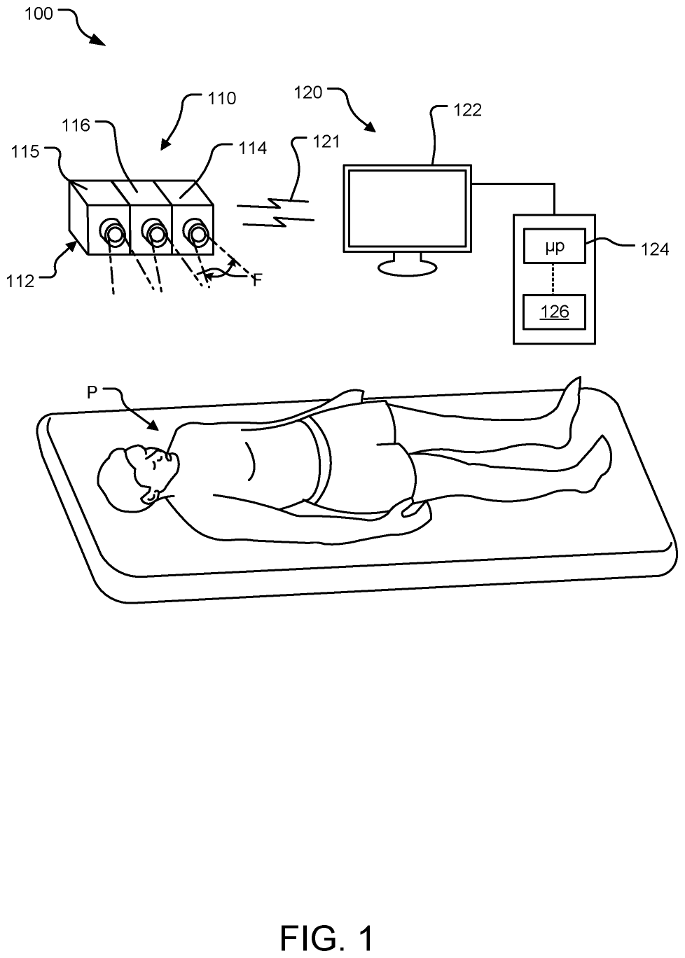

FIG. 1 shows a non-contact patient monitoring system 100 and a patient P. The system 100 includes a non-contact detector system 110 placed remote from the patient P. In this embodiment, the detector system 110 includes a camera system 112, particularly, that includes an infrared (IR) detection feature. The camera system 112 includes a first camera 114 and a second camera 115, at least one of which is a depth sensing camera, such as a Kinect camera from Microsoft Corp. (Redmond, Wash.) or a RealSense™ D415, D435 or D455 camera from Intel Corp. (Santa Clara, Calif.).

The cameras 114, 115 are positioned so that their ROI at least intersect, in some embodiments overlap. The detector system 110 also includes an IR projector 116, which projects individual features (e.g., dots, crosses or Xs, lines, or a featureless pattern, or a combination thereof etc.) onto the ROI. The projector 116 can be separate from the camera system 112 or integral with the camera system 112, as shown in FIG. 1. In some embodiments, more than one projector 116 can be used. Both cameras 114, 115 and the projector 116 are aimed to have the features projected by the projector 116 to be in the ROI.

The cameras 114, 115 and projector 116 are remote from the patient P, in that they are spaced apart from and do not contact the patient P. The camera system 112 includes a detector exposed to a field of view F that encompasses at least a portion of the patient P.

The camera system 112 includes at least one depth sensing camera, either or both camera 114 or camera 115, that can detect a distance between the camera system 112 and objects in its field of view F. Such information can be used to determine that a patient is within the field of view of the camera system 112 and determine a region of interest (ROI) to monitor on the patient. Once an ROI is identified, that ROI can be monitored over time, and the change in depth of points within the ROI can represent movements of the patient associated with, e.g., breathing. Accordingly, those movements, or changes of depth points within the ROI, can be used to determine, e.g., respiration rate, tidal volume, minute volume, effort to breathe, etc.

In some embodiments, the field of view F encompasses exposed skin of the patient. In other embodiments, the field of view F encompasses a portion of the patient's torso, covered by a blanket, sheet, or gown.

The cameras 114, 115 operate at a frame rate, which is the number of image frames taken per second (or other time period). Example frame rates include 15, 20, 30, 40, 50, or 60 frames per second, greater than 60 frames per second, or other values between those. Frame rates of 20-30 frames per second produce useful signals, though frame rates above 100 or 120 frames per second are helpful in avoiding aliasing with light flicker (for artificial lights having frequencies around 50 or 60 Hz).

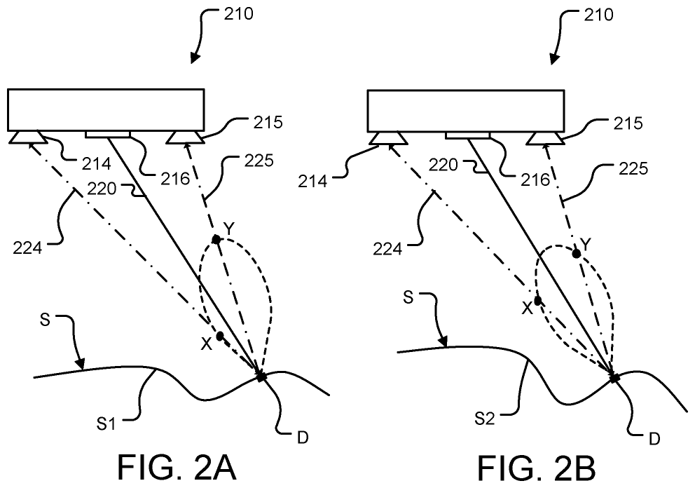

The distance from the ROI on the patient P to the camera system 112 is measured by the system 100. Generally, the camera system 112 detects a distance between the camera system 112 and the projected features on the surface of the patient P within the ROI; the change in depth or distance of the ROI represents movements of the patient P, e.g., associated with breathing. The light from the projector 116 hitting the surface is scattered/diffused in all directions and is monitored by the camera system 112 to determine the distance; the diffusion pattern depends on the reflective and scattering properties of the surface. The camera system 112 also detects the light intensity of the projected individual features in their ROIs. From the distance and the light intensity, at least one physiological parameter of the patient P is monitored. Additional details are provided below in respect to FIG. 2A and FIG. 2B.

In some embodiments, the system 100 determines a skeleton outline of the patient P to identify a point or points from which to extrapolate the ROI. For example, a skeleton may be used to find a center point of a chest, shoulder points, waist points, and/or any other points on a body. These points can be used to determine the ROI. For example, the ROI may be defined by filling in the area around a center point of the chest. Certain determined points may define an outer edge of an ROI, such as shoulder points. In other embodiments, instead of using a skeleton, other points are used to establish an ROI. For example, a face may be recognized, and a chest area inferred in proportion and spatial relation to the face. In other embodiments, the system 100 may establish the ROI around a point based on which parts are within a certain depth range of the point. In other words, once a point is determined that an ROI should be developed from, the system can utilize the depth information from the depth sensing camera system 112 to fill out the ROI as disclosed herein. For example, if a point on the chest is selected, depth information is utilized to determine the ROI area around the determined point that is a similar distance from the depth sensing camera 114 as the determined point. This area is likely to be a chest.

In another example, the patient P may wear a specially configured piece of clothing that identifies points on the body such as shoulders or the center of the chest. The system 100 may identify those points by identifying the indicating feature of the clothing. Such identifying features could be a visually encoded message (e.g., bar code, QR code, etc.), or a brightly colored shape that contrasts with the rest of the patient's clothing, etc. In some embodiments, a piece of clothing worn by the patient may have a grid or other identifiable pattern on it to aid in recognition of the patient and/or their movement. In some embodiments, the identifying feature may be stuck on the clothing using a fastening mechanism such as adhesive, a pin, etc. For example, a small sticker or other indicator may be placed on a patient's shoulders and/or center of the chest that can be easily identified from an image captured by a camera. In some embodiments, the indicator may be a sensor that can transmit a light or other information to the camera system 112 that enables its location to be identified in an image so as to help define the ROI. Therefore, different methods can be used to identify the patient and define an ROI.

The ROI size may differ according to the distance of the patient from the camera system. The ROI dimensions may vary linearly with the distance of the patient from the camera system. This ensures that the ROI scales according with the patient and covers the same part of the patient regardless of the patient's distance from the camera. This is accomplished by applying a scaling factor that is dependent on the distance of the patient (and the ROI) from the camera. In order to properly measure the depth changes, the actual size (area) of the ROI is determined and movements of that ROI are measured. The measured movements of the ROI and the actual size of the ROI are then used to calculate the respiratory parameter, e.g., a tidal volume. Because a patient's distance from a camera can change, e.g., due to rolling or position readjustment, the ROI associated with that patient can appear to change in size in an image from a camera. However, using the depth sensing information captured by a depth sensing camera or other type of depth sensor, the system can determine how far away from the camera the patient (and their ROI) actually is. With this information, the actual size of the ROI can be determined, allowing for accurate measurements of depth change regardless of the distance of the camera to the patient.

In some embodiments, the system 100 may receive a user input to identify a starting point for defining an ROI. For example, an image may be reproduced on an interface, allowing a user of the interface to select a patient for monitoring (which may be helpful where multiple humans are in view of a camera) and/or allowing the user to select a point on the patient from which the ROI can be determined (such as a point on the chest). Other methods for identifying a patient, points on the patient, and defining an ROI may also be used.

To determine the distance from the camera system 112 and the projected image on the patient P, the detected images and diffusion measurements (detected by the camera system 112) are sent to a computing device 120 through a wired or wireless connection 121. The computing device 120 includes a display 122, a processor 124, and hardware memory 126 for storing software and computer instructions. Sequential image frames of the patient P are recorded by the video camera system 112 and sent to the processor 124 for analysis. The display 122 may be remote from the camera system 112, such as a video screen positioned separately from the processor and memory. Other embodiments of the computing device 120 may have different, fewer, or additional components than shown in FIG. 1. In some embodiments, the computing device may be a server. In other embodiments, the computing device of FIG. 1 may be additionally connected to a server. The captured images (e.g., still images, or video) can be processed or analyzed at the computing device and/or at the server to determine the parameters of the patient P as disclosed herein.

FIG. 2A and FIG. 2B both show a non-contact detector 210 having a first camera 214, a second camera 215, and an IR projector 216. A dot D is projected by the projector 216 onto a surface S, e.g., of a patient, via a beam 220. Light from the dot D is reflected by the surface S and is detected by the camera 214 as beam 224 and by the camera 215 as beam 225.

In a particular implementation, the light intensity returned to and observed by the cameras 214, 215 depends on the diffusion pattern caused by the surface S (e.g., the surface of a patient), the distance between the cameras 214, 215 and surface S, the surface gradient, and the orientation of the cameras 214, 215 relative to the surface S. In FIG. 2A, the surface S has a first profile S1 and in FIG. 2B, the surface S has a second profile S2 different than S1; as an example, the first profile S1 is during an exhale breath of a patient and the second profile S2 is during an inhale breath of the patient. Because the surface profiles S1 and S2 differ, the deflection pattern from the dot D on each of the surfaces differs for the two figures, and hence the distance from the cameras 214, 215 to the surface differs for the two figures.

During breathing (respiration), the light intensity reflection off the dot D observed by the cameras 214, 215 changes because the surface profile S1 and S2 (specifically, the gradient) changes as well as the distance between the surface S and the cameras 214, 215. FIG. 2A shows the surface S having the surface profile S1 at time instant t=tₙ and FIG. 2B shows the surface S having the surface profile S2 at a later time, specifically t=tₙ₊₁, with S2 being slightly changed due to motion caused by respiration. Consequently, the intensity of the projected dot D observed by the cameras 214, 215 will changed due to the changes of the surface S. In FIG. 2A, a significantly greater intensity is measured by the camera 215 than the camera 214, seen by the x and y on the beams 224, 225, respectively. In FIG. 2B, y is less than y in FIG. 2A, whereas x in FIG. 2B is greater than x in FIG. 2A. The manner in how these intensities change depends on the diffusion pattern and its change over time, which are related to movement of the surface S. As seen in FIGS. 2A and 2B, the light intensities as measured by the cameras 214, 215 have changed between FIGS. 2A and 2B, and hence, the surface S has moved. Each camera will generate a signal because of the change of the intensity of dot D when the surface profile changes from time instant t=tₙ to t=tₙ₊₁ due to movement.

In some other embodiments, a single camera and light projector can be used. For example, the camera 215 may be not present or is ignored. It is clear that the camera 214 will still produce a change in light intensity from time instant t=tₙ to t=tₙ₊₁ due to movement. This embodiment will therefore produce only a single signal as opposed to the two signals generated by the embodiment discussed in the previous paragraph.

Alternatively, other depth camera detectors may be used for the monitoring system. For example, the depth camera detector and/or the depth camera(s) may be based on, for example, stereoscopic, structured light, or time-of-flight principles.

Stereoscopic depth cameras resolve depth by using two slightly different perspective views of the same scene, similar to the detector 210 of FIGS. 2A and 2B; this is similar to the manner in which frontal vision animals perceive depth. Algorithmically, the depth data is constructed from the two views by calculating the disparities between features or key points in the scene.

Structured light and related coded light-based cameras project a pattern (e.g., an IR pattern) onto a scene; the pattern, which may be a series of stripes or dots, for example, has a known visual shape. Depth data is obtained by analyzing the deformation of the shape perceived by the camera, the deformation due to the movement of the scene. This detected movement is correlated to the distance from the cameras to the deformed pattern on the scene.

Depth cameras operate on the time-of-flight principle and measure distance (depth) to points in the scene by measuring the time it takes for a signal emitted from the camera to return due to reflection from a surface. The scene is actively illuminated by the camera's emitter (e.g., a radiation emitter, such as an IR laser) and the camera recovers the distance information either through a direct (i.e., half the return time) or indirect (i.e., phase recovery of a modulated emitted signal) method.

In addition to the methods and cameras/detectors described above, any suitable method for determining depth data from a scene can be used in the methods described herein.

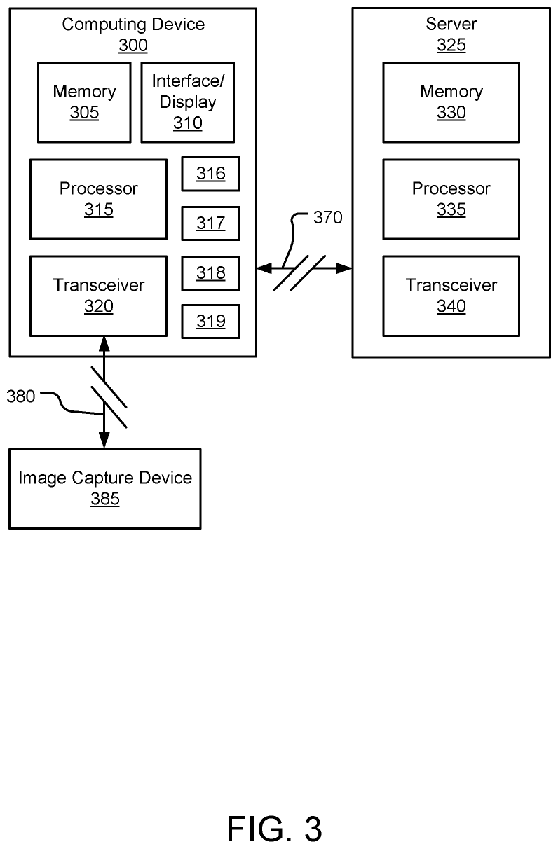

FIG. 3 is a block diagram illustrating a system including a computing device 300, a server 325, and an image capture device 385 (e.g., a camera, e.g., the camera system 112 or cameras 114, 115). In various embodiments, fewer, additional and/or different components may be used in the system.

The computing device 300 includes a processor 315 that is coupled to a memory 305 to store and recall data and applications in the memory 305, including applications that process information and send commands/signals according to any of the methods disclosed herein. The computing device 300 includes, in this example, modules 316, 317, 318 and 319, each configured to execute one or more of the analytical methods for manipulating the depth data described below to determine the mask for the ROI.

The processor 315 may also display objects, applications, data, etc. on an interface/display 310. The processor 315 may also or alternately receive inputs through the interface/display 310. The processor 315 is also coupled to a transceiver 320. With this configuration, the processor 315, and subsequently the computing device 300, can communicate with other devices, such as the server 325 through a connection 370 and the image capture device 385 through a connection 380. For example, the computing device 300 may send to the server 325 information determined about a patient from images captured by the image capture device 385, such as depth information of a patient in an image.

The server 325 also includes a processor 335 that is coupled to a memory 330 and to a transceiver 340. The processor 335 can store and recall data and applications in the memory 330. In some implementations, the server 325 may include the modules for manipulating the depth data, rather than the computing device 300. With this configuration, the processor 335, and subsequently the server 325, can communicate with other devices, such as the computing device 300 through the connection 370.

The computing device 300 may be, e.g., the computing device 120 of FIG. 1. Accordingly, the computing device 300 may be located remotely from the image capture device 385, or it may be local and close to the image capture device 385 (e.g., in the same room). The processor 315 of the computing device 300 may perform any or all of the various steps disclosed herein. In other embodiments, the steps may be performed on a processor 335 of the server 325. In some embodiments, the various steps and methods disclosed herein may be performed by both of the processors 315 and 335. In some other embodiments, certain steps may be performed by the processor 315 while others are performed by the processor 335. Information determined by the processor 315 may be sent to the server 325 for storage and/or further processing.

The devices may be utilized in various ways. For example, either or both of the connections 370, 380 may be varied. For example, either or both the connections 370, 380 may be a hard-wired connection. A hard-wired connection may involve connecting the devices through a USB (universal serial bus) port, serial port, parallel port, or other type of wired connection to facilitate the transfer of data and information between a processor of a device and a second processor of a second device. In another example, one or both of the connections 370, 380 may be a dock where one device may plug into another device. As another example, one or both of the connections 370, 380 may be a wireless connection. These connections may be any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another example, the various devices may connect through an internet (or other network) connection. That is, one or both of the connections 370, 380 may represent several different computing devices and network components that allow the various devices to communicate through the internet, either through a hard-wired or wireless connection. One or both of the connections 370, 380 may also be a combination of several modes of connection.

The configuration of the devices in FIG. 3 is merely one physical system on which the disclosed embodiments may be executed. Other configurations of the devices shown may exist to practice the disclosed embodiments as well as configurations of additional or fewer devices than the ones shown in FIG. 3. Additionally, any of the devices shown in FIG. 3 may be combined to allow for fewer devices than shown or separated such that more than the three devices exist in a system. It will be appreciated that many various combinations of computing devices may execute the methods and systems disclosed herein. Examples of such computing devices may include other types of medical devices and sensors, infrared cameras/detectors, night vision cameras/detectors, other types of cameras, radio frequency transmitters/receivers, smart phones, personal computers, servers, laptop computers, tablets, RFID enabled devices, or any combinations of such devices.

The method of this disclosure utilizes depth (distance) information between the camera(s) and the patient to determine a respiratory parameter such as respiratory rate. A depth image or depth map, which includes information about the distance from the camera(s) to each point in the image, can be measured or otherwise captured by a depth sensing camera, such as a Kinect camera from Microsoft Corp. (Redmond, Wash.) or a RealSense™ D415, D435 or D455 camera from Intel Corp. (Santa Clara, Calif.) or other sensor devices based upon, for example, millimeter wave and acoustic principles to measure distance. The depth image or map can be obtained by a stereo camera, a camera cluster, camera array, or a motion sensor focused on a ROI, such as a patient's chest. In some embodiments, the camera(s) are focused on visible or IR features in the ROI. Each projected feature may be monitored, less than all the features in the ROI may be monitored or all the pixels in the ROI can be monitored.

Because the image or map includes depth data from the depth sensing camera(s), information on the spatial location of the patient (e.g., the patient's chest) in the ROI can be determined. As the patient breathes, the patient's chest moves toward and away from the camera, changing the depth information associated with the images over time. As a result, the location information associated with the ROI changes over time. The position of individual points within the ROI (e.g., the change in distance) may be integrated across the area of the ROI to provide a change in volume over time.

For example, movement of a patient's chest toward a camera as the patient's chest expands forward represents inhalation. Similarly, movement backward, away from the camera, occurs when the patient's chest contracts with exhalation. This movement forward and backward can be tracked to determine a respiration rate. The respiration rate can be integrated to determine respiration volume.

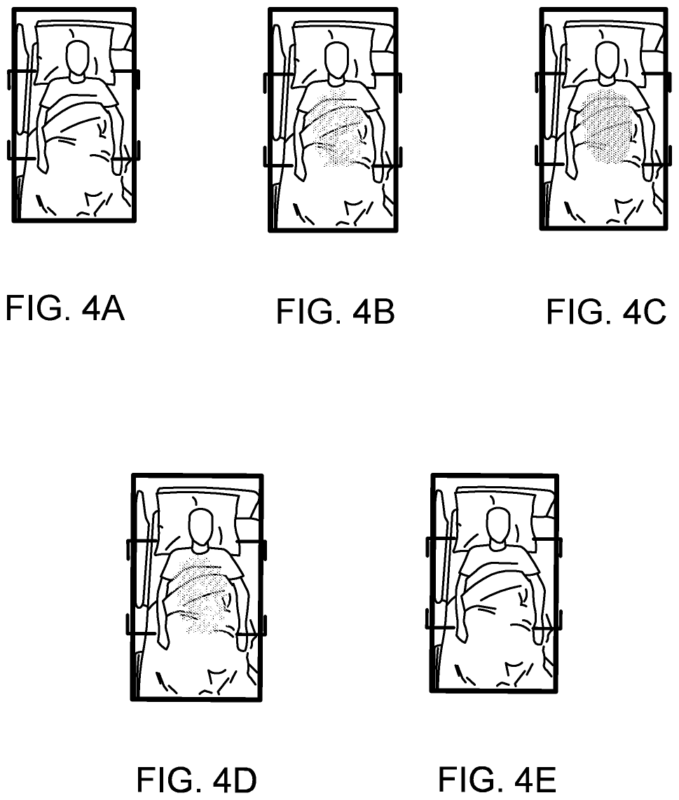

FIGS. 4A through 4E illustrate forward and backward movement of a chest area of a patient, and thus, respiration by the patient. FIGS. 4A through 4E show one breath sequence. In these figures, the ROI region is moving towards the camera (e.g., on an inhale) in FIG. 4B, it at its maximum in FIG. 4C, and is moving away from the camera (e.g., on an exhale) in FIG. 4D.

Depending on the system parameters, the forward and backward movement can be evidenced by a color change applied by the monitoring system. For example, when the ROI region is moving towards the camera (e.g., on an inhale), a green overlay can be shown, whereas when the ROI region is moving away from the camera (e.g., on an exhale), no color overlay is shown. In other implementations, the user or viewer of the monitoring system can select the settings of the visual output. For example, the user may desire a green overlay for an inhale and a red overlay for an exhale, or, a white overlay for an inhale and no color overlay for an exhale, e.g., for user that are red/green colorblind. In some arrangements, the strength, tone, or brightness of the selected color may change as the movement (e.g., distance) changes.

In some instances, there is noise in the depth data, whether noise from the ROI environment or from the camera(s). With sufficiently accurate cameras and in environments with little noise, the overlay is visually coherent, as seen in FIGS. 4A through 4E, allowing for readily discernable respiration. However, in some applications, noise in the ROI yet outside of the chest area of the patient affects the coherency of the overlay, hindering a distinct and/or clear overlay.

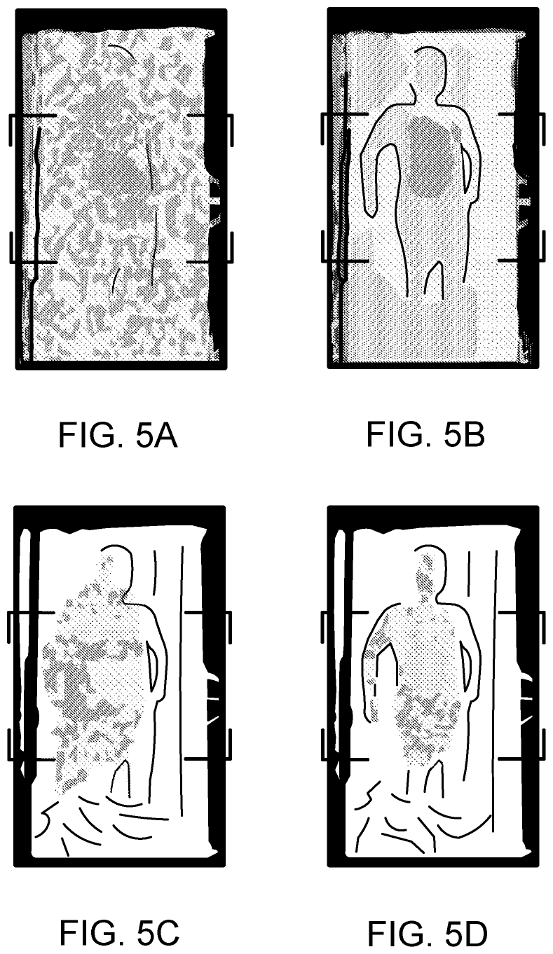

For example, regions in the ROI that should appear stationary (e.g., a static floor or a bed) can appear to be moving due to noise in the ROI. An example of noise is seen in FIG. 5A. When conditions are poor, this noise can be of the same magnitude as the actual physiological modulations in the scene (e.g., caused by respiration). Provided herein are methods for identifying regions of the ROI that are respiration and generating a “respiration mask,” the mask then being used to identify and emphasize the regions of the ROI where respiration occurs, avoiding regions where respiration does not occur. By using such a mask, the effect of noise on the final product (image) provided to the user is reduced.

FIGS. 5A through 5D show four levels of masking in the ROI regions indicated by the brackets. FIG. 5A and FIG. 5B show two examples of having no mask and where the noise is readily seen on the bed on which the patient is lying. FIG. 5C shows a single mask, referred to as “Corrmask” below, applied to the patient respiration image. A fully composite mask is applied to the patient image in FIG. 5D. This full mask is a cumulation of the “Corrmask,” the “Spatial coherence mask” below, and various thresholding and averaging steps, including a “pseudo-mask” generated by applying a contrast limited adaptive histogram equalization (CLAHE) transform to the depth data, so that the transformed data has zero values in the flat regions of the image (e.g., the bed). The improvement in overlay is readily seen by comparing FIG. 5A to FIG. 5D.

The respiration mask may be determined by numerous methods, some examples of which are provided below. In each of the examples, the depth data in the ROI are examined to determine the regions of the ROI that correspond to respiration. The ROI is a region of interest within the whole field of view (FOV). The respiration mask limits the monitored ROI area to those regions having respiration, avoiding areas that are not respiration and thus that should be avoided in the respiration calculation. In some implementations, the entire FOV may be monitored, but only the areas within the respiration mask are used for the respiration calculation. The region to which the mask is applied is referred to herein as the depth field; the depth field can be the entire FOV or an ROI within the FOV.

In general, a respiration mask is determined by from the depth data by any method or multiple methods. After determining the mask, it is known which regions of the depth field correspond to respiration. The mask is used to extract the depth data from only the regions corresponding to respiration; from this, a visual overlay, which can be color coded to represent moving toward the camera (inhale) and moving away from the camera (exhale). The original depth data in the depth field can also be used to create a background image (e.g., of the patient, bed etc.); this can be done, e.g., by applying histogram equalization and then a visual color map, or, by applying first a visual color map and then applying histogram equalization). The background image can be combed with the mask overlay; this provides the user with a view of the patient with the respiration overlayed on top.

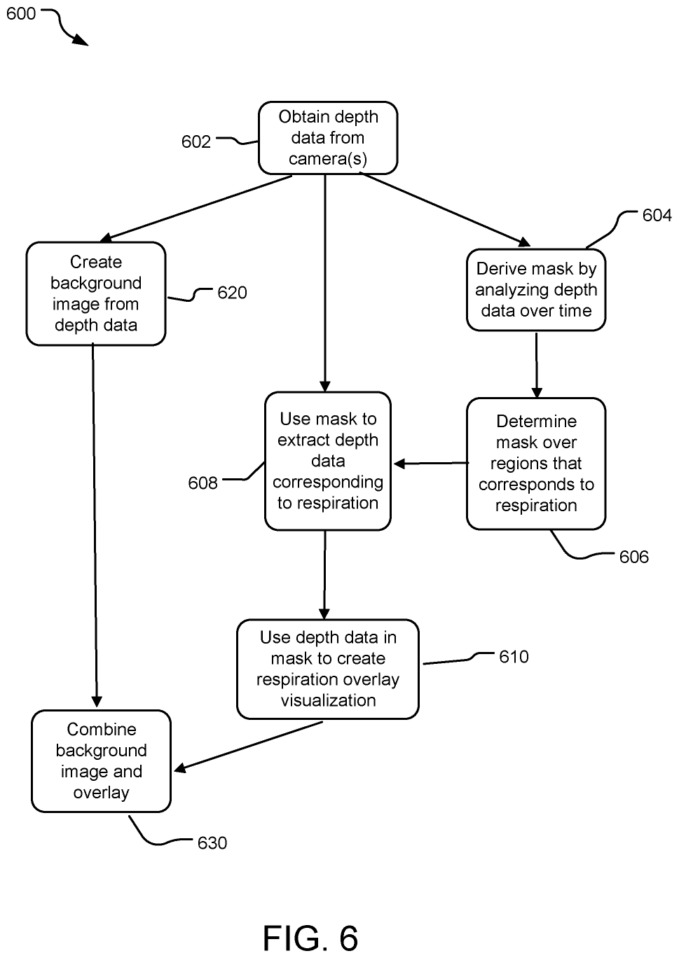

FIG. 6 shows, stepwise, an overall method 600 for determining and applying a mask to a respiration image from a non-contact monitoring system (e.g., system 100 of FIG. 1). The mask is determined from depth data and is overlayed on an image of the patient also generated from the depth data.

In step 602, depth data from camera(s) of a non-contact monitoring system is obtained from a patient. The monitoring is done via depth data obtained from depth camera(s) based on the distance of the patient or other surface in relation to the depth camera(s).

In step 604, the mask is derived by analyzing the depth data over time, e.g., by one or more of the methods described herein, to determine which of the monitored regions possible represent respiration. In step 606, the mask is limited to those regions that correspond to respiration. This may be done by, e.g., manipulating the depth data (e.g., filtering, thresholding, smoothing, transforming, etc.) to determine the respiration regions to create the mask defining the respiration image.

In step 608, this mask from step 606, is used to extract the depth data in the ROI that possibly corresponds to the patient's respiration. In some implementations, this step 608 can be combined with a previously determined mask.

From the mask from step 608, depth data from the mask is used to create a respiration overlay visualization (e.g., as seen in FIG. 5D) in step 610.

Simultaneously to creating the mask (steps 604, 606, 608) and creating the overlay visualization (step 610), in step 620, a visual image of the patient and the background (e.g., as seen in FIG. 5D) is created from the depth data.

In step 630, the patient and background image from step 620 is combined with the respiration overlay visualization from step 610.

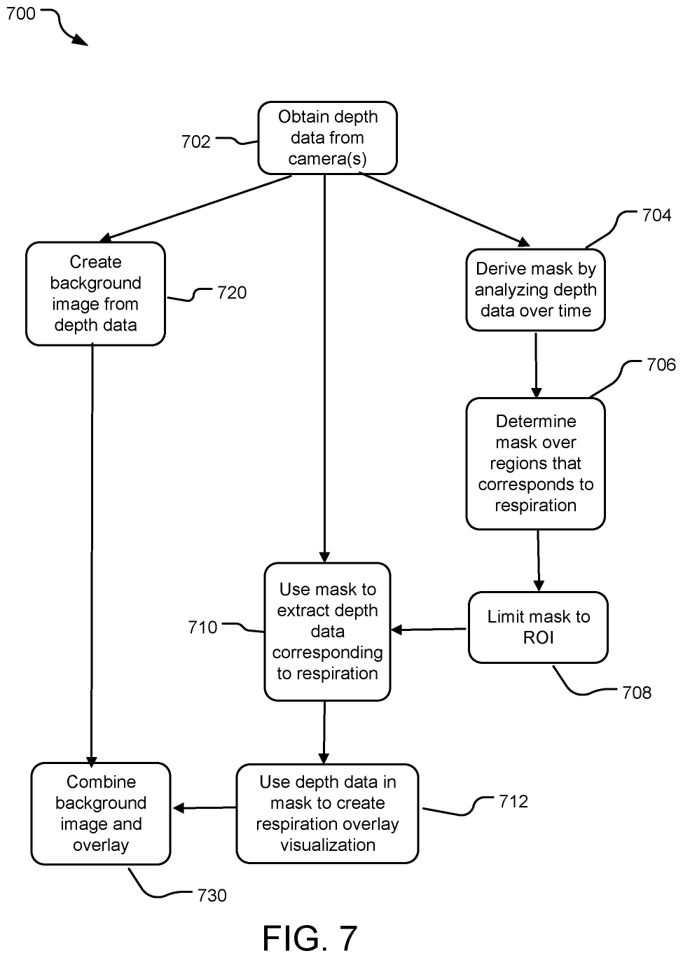

Another overall method for determining and applying a mask to a respiration image from a non-contact monitoring system (e.g., system 100 of FIG. 1) is shown in FIG. 7 as method 700. As in the method 600, the mask is determined from depth data and is overlayed on an image of the patient also generated from the depth data, but here is limited to a predetermined ROI.

In step 702, depth data from camera(s) of a non-contact monitoring system is obtained from a patient. The monitoring is done via depth data obtained from depth camera(s) based on the distance of the patient or other surface in relation to the depth camera(s).

In step 704, the mask is derived by analyzing the depth data over time, e.g., by one or more of the methods described herein, to determine which of the monitored regions possible represent respiration. In step 706, the mask is limited to those regions that correspond to respiration. This may be done by, e.g., manipulating the depth data (e.g., filtering, thresholding, smoothing, transforming, etc.) to determine the respiration regions to create the mask defining the respiration image.

In step 708, the mask is limed to the regions defined by a hard coded ROI (see the ROI brackets in, e.g., FIG. 5B). Thus, the mask does not extend farther than the ROI.

In step 710, this mask from step 708, is used to extract the depth data in the ROI that possibly corresponds to the patient's respiration. In some implementations, this step 710 can be combined with a previously determined mask.

From the mask from step 710, depth data from the mask is used to create a respiration overlay visualization (e.g., as seen in FIG. 5D) in step 712.

Simultaneously to creating the mask (steps 704, 706, 708, 710) and creating the overlay visualization (step 712), in step 720, a visual image of the patient and the background (e.g., as seen in FIG. 5D) is created from the depth data.

In step 730, the patient and background image from step 720 is combined with the respiration overlay visualization from step 712.

In alternate methods, the image creation steps 620, 720 may be replaced with a step that utilizes an infrared (IR) image or an RGB image to create the patient and background image instead of depth data.

As indicated above, in the method 600 the depth data are examined to determine the regions of the ROI that correspond to respiration, whereas in method 700 the depth data in the ROI are examined to determine the regions of the ROI that correspond to respiration. The depth data in these regions may be examined and/or evaluated by any numerous methods to determine the location and properties of the mask applied to the ROI to emphasize the respiration.

Example 1: Corrmask. In this example, referred to as “Corrmask” herein, a correlated mask is generated based on the local relationship between the depth gains and losses (i.e., moving towards and away from the camera(s), respectively).

The Corrmask can be calculated from the gains and losses in multiple regions of the depth of field being mathematically manipulated to determine a long term average of each region. The long term average of each region is then used to determine the regions of respiration (the mask). A zero or close to zero long term average indicates that the region does not have respiration and are thus removed from the mask region. The remaining regions are evaluated against a mean value of all the remaining regions. Regions having a long term average value greater than the mean are re-valued at the mean value. One or more histogram equalizations is performed on the regions; multiple histogram equalizations may be done in parallel or sequentially. The results may be further manipulated (e.g., upscaled, downscaled) to highlight the depth gains and losses, thus highlighting respiration.

Example 2: Spatial Coherence Mask. In this example, referred to as “Spatial Coherence mask” herein, the mask is generated based on the spatial coherence of the change in depth data. This method relies on the fact that different regions of the depth field behave differently, particularly, that for respiration, the changes in the depth field are coherent (e.g., they tend to move in the same direction, towards or away from the camera(s)), and for background or a stationary surface, the changes in the depth field are incoherent (e.g., they tend to have a “zero” center). The noise in the depth field cancels itself out over a large enough window.

The Spatial Coherence mask can be calculated by calculating a recent change in depth for each of multiple regions of the depth field and assigning one of two binary values based on the direction of depth change, ignoring the magnitude of the change. A filter (e.g., box filter) is applied to the values and the local bias in the depth field is calculated to effectively a measure of how coherent the changes are in the depth field surrounding each point in the depth field. If the noise is incoherent, the local bias is small (e.g., close to 0), compared to a region where the torso is clearly moving towards or away from the camera(s), the local bias is large (e.g., close to 1). Small values (e.g., with a cut off at e.g., less than 0.2 or less than 0.4) can be removed from the local bias and the resulting trimmed local bias mask can be used to highlight respiration in the depth field as the Spatial Coherence mask.

In an alternate method of determining a Spatial Coherence mask, the mask can be calculated for every depth data received from the camera (e.g., at 15 frames per second, or 30 frames per second) using the calculation outlined above. The trimmed bias mask from above can be combined with any number of previous mask(s) from the previous frame(s).

A property of these Spatial Coherence masks, whether the instant mask of the first calculation or the combined mask of the second calculation, is that it is likely to be empty when the patient's chest is stationary (e.g., at the top or bottom of a breath). Because of this, care should be taken to prevent the mask from being intermittent. To avoid an intermittent mask, the mask is created as described above for the Spatial Coherence mask but then the regions are monitored for change. If the total of the mask regions are not empty but have a value (e.g., more than 1% of them are not empty), the mask is added to the previous mask (e.g., to the instant mask or the combined mask). Subsequent masks are added to the previous mask. Having cumulative sequential masks inhibits the long-term mask from disappearing.

To inhibit a lingering or stale mask, one that does not react when the patient's respiration has changed (e.g., when breathing stops), when the mask age is greater than a threshold (e.g., 15, 30, 60, 120 frames, or, e.g., 0.5, 1, 2, 3 seconds) then the mask is reset.

In another alternate method of calculating a Spatial Coherence mask, a mask is generated at multiple scales. Different sized filters can be used when calculating the local bias and then the different masks can be combined into a single mask.

Example 3: XOR Mask. In this example, referred to as “XOR mask” or “XOR” herein, the mask is generated by exploiting the various properties of the depth field. XOR is a logical operation that returns true only if the two inputs do not agree. When a patient is breathing, large regions of the torso move towards or away from the camera(s) in a coherent fashion. In regions without respiration, the gains and losses are more likely to be incoherent and are due to noise in the depth field measurement.

An XOR mask can be calculated by applying a spatial box filter to the gain and to the loss of each region (e.g., a filter width of 5, 10, 25, etc.) to generate a smoothed loss and smoothed gain and then applying a threshold to the smoothed loss/gain. The threshold highlights a tendency in a region for a movement towards or a movement away from the camera(s). In the absence of noise, these fields would be the opposite of one another; that is, in any region with a gain, there would be no loss. However, the noise in the depth field means that the opposite values (gains and losses) only exist in the region of the depth field having respiration. The movement of the torso due to respiration means that the gain and losses are coherent.

Example 4: FFT Mask. In this example, the mask is generated by analyzing the FFT (fast Fourier transform) of the relative change in depth for each region in the depth field, and the mask is thus referred to as an “FFT mask.” For the FFT mask, each region is defined as a single pixel or as a number of pixels (e.g., a 16×16 grid across the depth field). When a region is made up of multiple pixels, the multiple pixels can be combined using any suitable method, such as taking the mean, the median, a trimmed mean, etc.

An FFT mask can be calculated by applying suitable filtering (e.g., a Low Pass filter that overlaps the physiologically plausible respiratory frequencies) to a 1-D time-series of depth changes for each region. A discrete Fourier transform (DFT) is performed on each of the time-series to determine the frequencies present in each region and a dominant frequency is identified. The power (N) contained around the dominant frequency is calculated across a fixed number of bins around the central frequency, as is well known. The ratio of the power at that frequency over the power elsewhere is used to determine the FFT mask.

Described above are various methods for preparing masks to inhibit noise from non-contact, remote, respiratory monitoring data. Some of the individual masks (e.g., a Corrmask, or a bias mask) may have false positives (regions lit up that should be hidden) or false negatives (regions that should be lit up but are not). Each mask, depending on the method to create the mask, will have different performance characteristics; one mask may be more likely to produce better results in a particular scenario than another.

Any of the masks described above (e.g., Corrmask, XOR mask, bias mask, etc.), and variations thereof, may be applied to the depth field individually, or multiple masks may be run in parallel or in sequence during monitoring, or multiple masks may be combined to create a superior mask. Multiple masks may be combined in a number of ways, e.g., majority voting, mean, weighted mean, weighted sum, etc., to provide a combined mask. The combined mask can be further processed, e.g., by contour/blob analysis to remove noise and/or spurious regions to provide a final superior mask.

As mentioned above, some of the individual masks may have false positives or false negatives, depending on the mask and the environment it is used. By combining the masks to create a combined mask and then a superior mask based on iterative calculations, these false readings are reduced compared to a single mask.

Each individual mask can be a matrix where zero values correspond to “no respiration” and non-zero values correspond to a measure of the likelihood or the strength of the respiration present at that pixel (or region) in the image. The larger the value at a pixel or region, the greater the likelihood of respiration. As such, multiple masks can be combined using any suitable method.

For example, multiple masks can be combined via a modified geometric mean, optionally from which an offset is subtracted. In another example, the masks at each pixel can be averaged to determine the combined mask. In another example, a power law weighting can be used. To account for variability in the relative magnitudes of the individual masks, a multiplication factor (e.g., K) may also be applied to each mask values. In other examples, the median value or any other percentile value of the combined individual masks may be used as the combined mask. In another example, the combined mask may be based on only some of the individual masks, e.g., only the 2 or 3 or 4 largest individual masks (depending on the total number of individual masks available), only half of the individual masks, only the largest half of the individual masks. The resulting combined mask may be, e.g., the mean or median value of the selected masks. In some calculations, the respiratory waveform obtained from each individual mask can be used to determine the weighting of each mask; those that produce relatively poorer respiratory waveforms can be down weighted, e.g., in the averaging or other combining of the masks.

In some implementations, once a combined mask is obtained, the combined mask can be further refined, e.g., focusing the mask on “good” regions and avoiding “bad” regions. “Good” regions can be identified by assigning upper and/or lower threshold(s), which can be done by various methods. For example, the threshold can be a percentile of the values in the mask, e.g., the 25^{th}, the 50^{th}, the 75^{th}, etc. percentile. In one implementation, the threshold may be a scaled percentile of the values in the mask; for example, the threshold may be 60% of the 75^{th} percentile. In another implementation, the threshold (e.g., lower threshold) can be comparative; for example, 60% of the 75^{th} percentile, or, the mean value, whichever is greater. In another example, a threshold may be ascertained from a region of the image where there is no noted respiration. A characteristic value of this region may be taken as the threshold. This may be a percentile of the (background) region's values, or may include values above the background region values, e.g., the threshold could be set to 1.5× or 2× the (background) region characteristic value. Once a threshold is determined, by any manner, a binary mask can be defined, by setting values below the threshold to 0 (zero) and values above the threshold to 1.

Additionally or alternately, “bad” regions can be removed from the combined mask by analyzing the regions and discarding obviously incorrect regions. This is particularly beneficial and straightforward for a binary mask. For example, in all regions of the mask, the number of features in each region is analyzed and the values are combined into a score. Regions having a low score are discarded from the mask. A low score can be relative to, e.g., the mean of the values in the region, the maximum value in the region, the minimum value in the region, the area of the region (e.g., either as a percentage, or in actual units (e.g., mm, m), or pixel units), the perimeter of the region, the circularity of the region, etc.

By applying a histogram equalization technique (e.g., contrast limited adaptive histogram equalization, or CLAHE) to the depth data, regions that are unlikely to have respiration are “zeroed” out. Such is a basic method for creating a mask that can be referred to as a transformed-depth, or a modified-depth, or a depth-clahe-mask, or a pseudo mask.

Any one or more of the masks, which highlight or emphasize the regions of the image that contain respiration, can be applied to the image to improve the output image for the user. See, for example, FIG. 5D and compare to FIG. 5A.

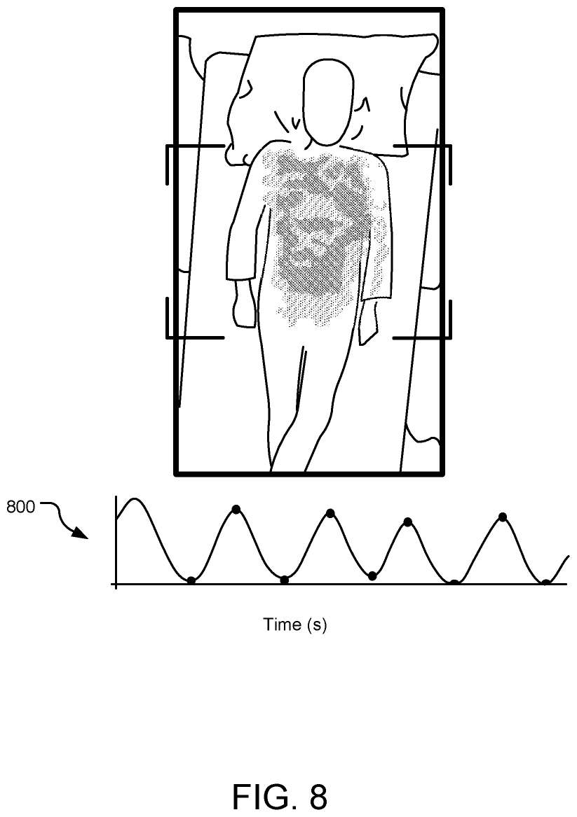

FIG. 8 shows an example of the respiratory waveform 800 generated from a mask on a patient P, showing the respiration of the patient P. The quality of the waveform may be determined using a metric which captures the repeatability of the waveform, or its smoothness, etc. Not only can a mask smooth the data, providing a coherent image and a smooth waveform for respiratory rate, but additional information can be extracted, for example, respiratory volume can be extracted from the region defined by the mask.

Thus, described herein are methods and systems for improving a visual output image from non-contact monitoring of a patient to determine respiratory parameters by applying a mask to the depth data to reduce and inhibit noise in the resulting output image.

The above specification and examples provide a complete description of the structure and use of exemplary embodiments of the invention. The above description provides specific embodiments. It is to be understood that other embodiments are contemplated and may be made without departing from the scope or spirit of the present disclosure. The above detailed description, therefore, is not to be taken in a limiting sense. For example, elements or features of one example, embodiment or implementation may be applied to any other example, embodiment or implementation described herein to the extent such contents do not conflict. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

1. A method of determining a respiratory parameter of a patient, comprising:
determining depth data between a non-contact patient monitoring system and the patient, over time, in a region of interest (ROI) on the patient, the ROI having multiple regions and each region having at least one depth data;
analyzing each of the multiple regions in the ROI to create a background image overlay from the depth data;
analyzing each of the multiple regions in the ROI to determine whether respiration is occurring in the analyzed region, and dependent on respiration not occurring in an analyzed region, not including that analyzed region in a mask calculation, and dependent on respiration occurring in an analyzed region, including that analyzed region in the mask calculation;
preparing a mask comprising a plurality of regions from the mask calculation and extracting the depth data corresponding to respiration;
using the depth data corresponding to respiration to create a visual respiration overlay; and
combining the visual respiration overlay with the background image overlay to produce a respiration image.

2. The method of claim 1, wherein determining the depth information comprises using at least one depth-sensing camera.

3. The method of claim 2, wherein determining the depth information comprises measuring a light intensity from the multiple regions with the at least one depth-sensing camera.

4. The method of claim 2, wherein determining the depth information comprises:
projecting a plurality of features onto the patient in the ROI;
measuring a first reflected light intensity from the features at a first time;
measuring a second reflected light intensity from the features at a second time subsequent to the first time; and
comparing the first reflected light intensity and the second reflected light intensity to determine a change in position of the features over time.

5. The method of claim 1, wherein preparing the mask comprising the plurality of regions from the mask calculation comprises:
preparing the mask comprising a plurality of regions from the mask calculation and combining the prepared mask with a previously prepared mask.

6. The method of claim 1, wherein analyzing each of the multiple regions in the ROI comprises calculating a gain and a loss for each region.

7. The method of claim 1, wherein analyzing each of the multiple regions in the ROI comprises applying a CLAHE transform to the depth data.

8. The method of claim 1, wherein analyzing each of the multiple regions in the ROI comprises:
recognizing that coherent changes in the depth data in a region represents respiration occurring; and
recognizing that incoherent changes in the depth data a region represents respiration not occurring.

9. The method of claim 8, further comprising:
assigning regions having coherent changes to the mask calculation; and
withholding regions having incoherent changes from the mask calculation.

10. The method of claim 1, wherein analyzing each of the multiple regions in the ROI and preparing a mask comprises:
applying a Fourier transform calculation to each of the multiple regions.

11. The method of claim 1, comprising:
preparing a second mask comprising the plurality of regions from a second mask calculation different than the mask calculation;
combining the mask and the second mask to form a combined mask; and
determining the respiratory parameter of the patient in the combined mask over the ROI.

12. The method of claim 1, wherein preparing a mask comprising a plurality of regions from the mask calculation further comprises limiting the mask to the ROI.

13. A method of determining a respiration region of interest (ROI) of a patient, comprising:
obtaining depth data from a non-contact patient monitoring system for a patient in a field of view (FOV);
analyzing the depth data from multiple regions in the FOV to create a background image from the depth data;
analyzing the depth data from the multiple regions the FOV to determine whether respiration is occurring, and dependent on respiration not occurring in an analyzed region, not including that analyzed region in a mask calculation, and dependent on respiration occurring in an analyzed region, including that analyzed region in the mask calculation;
preparing a mask from the mask calculation and extracting the depth data corresponding to respiration;
using the depth data corresponding to respiration to create a visual respiration overlay; and
combining the visual respiration overlay with the background image to produce a respiration image.

14. The method of claim 13 further comprising displaying the respiration image on a display screen.

15. The method of claim 13, wherein obtaining depth data from a non-contact patient monitoring system comprises using at least one depth-sensing camera.

16. The method of claim 13, wherein analyzing the depth data from multiple regions comprises calculating a gain and a loss for each region.

17. The method of claim 13, wherein analyzing the depth data from multiple regions comprises applying a CLAHE transform to the depth data.

18. The method of claim 13, wherein analyzing the depth data from multiple regions comprises:
recognizing that coherent changes in the depth data in a region represents respiration occurring; and
recognizing that incoherent changes in the depth data a region represents respiration not occurring,
the method further comprising:
assigning regions having coherent changes to the mask calculation; and
withholding regions having incoherent changes from the mask calculation.

19. The method of claim 13, comprising:
preparing a second mask comprising the plurality of regions from a second mask calculation different than the mask calculation;
combining the mask and the second mask to form a combined mask; and
determining the respiratory parameter of the patient in the combined mask.

20. The method of claim 13, wherein preparing a mask from the mask calculation further comprises limiting the mask to a region of interest (ROI).
